(19) United States
(12) Patent Application Publication
(10) Pub. No.: US 2008/0095822 A1
(43) Pub. Date: Apr. 24, 2008

(54) Active Substance Delivery System Comprising A Hydrogel Atrix And Microcarriers

(75) Inventors: Veronique MAQUET, Berioz (BE); Christophe PAGNOULLE, Verviers (BE); Brigitte EVRARD, Verlaine (BE); Robert JEROME, Jalhay (BE); Jean-Michel FOIDART, Trooz (BE); Francis FRANKENNE, Chaudfontaine (BE)

Correspondence Address: JACOBSON HOLMAN PLLC, 400 SEVENTH STREET N.W., SUITE 600, WASHINGTON, DC, 20004, US

(73) Assignee: UNIVERSITE DE LIEGE, Liege (BE)

(21) Appl. No.: 11/667,841

(22) PCT Filed: Nov. 4, 2005
(86) PCT No.: PCT/EP05/55751

§ 371 Date: May 16, 2007

(30) Foreign Application Priority Data
Nov. 16, 2004 (EP) 04105819.9

(51) Int. Cl. A61F 2/02

(52) U.S. Cl. 424/426

(57) ABSTRACT

Active substance delivery system, comprising a biocompatible and biostable hydrogel matrix, and biodegradable microcarriers which are homogenously embedded within the hydrogel matrix, and contain at least two active substances.

The present invention relates to a solid active substance delivery system, comprising:
a cross-linked hydrogel matrix, and
microcarriers which are embedded within the cross-linked hydrogel matrix. The microcarriers are made of biocompatible and biodegradable (co)polymer, are homogeneously embedded into the biocompatible cross-linked hydrogel matrix and contain at least two different active substances.

Active substance administration throughout local implantation of active substance delivery systems offers an unique possibility of delivering a (therapeutic) dose of an active substance, for example, a drug, for a certain period of time at a specific target site. Local administration, allows to reach a higher therapeutic index than systemic administration. As a consequence, the apparent drug efficiency is improved whereas side effects are lowered.

Local delivery can be achieved by either injection or implantation using different drug delivery technologies, ranging in size and geometry from nanoparticles to microspheres, semi-solids (hydro)gels to solid polymeric implants. Implantable devices are generally used for prolonged drug release duration and provide a more controlled release than injectable systems.

Solid polymer implants exist in the form of matrix or reservoir-type systems. In the matrix-type active substance delivery system, the active substance is homogeneously dispersed throughout the polymeric matrix. The active substance particles, present at the surface, firstly dissolve into the release medium, giving rise to a burst effect, creating a concentration gradient within the active substance delivery system that thermodynamically drives the release process. This release is thus a concentration-dependent release profile, non-constant over time. In the active substance delivery system comprising a reservoir or a core, the drug is located within a central core that is surrounded by a drug-free polymer membrane. In this case, the active substance is released in a zero-order fashion (release is constant over time) and controlled by the thickness of the membrane and core length. However, multiple active substance administration from a single reservoir active substance delivery system with different release profiles is hard to optimise since once the thickness of the sheet layer and core length have been chosen for one active substance or drug, they are irreversibly fixed for another.

In contraception and hormone replacement therapy, the release of two active substances in a substantially constant ratio to one another is frequently used. U.S. Pat. No. 4,596,576 describes a multi-compartments vaginal ring consisting of two or more reservoirs for the simultaneous release of several active substances. However, in order to keep constant the release ratio between the various active substances, each reservoir may be separated by stoppers (inert materials) making this device difficult to produce. U.S. Pat. No. 5,989,581 describes an intravaginal ring releasing progestogen and estrogen, simultaneously, in a fixed physiological ratio over a prolonged period of time. This device is made of a poly(ethylene-co-vinylacetate) (PEVA) core containing the mixture of hormones, the progestogen being dissolved in a relatively low degree of supersaturation. The core is surrounded by non-medicated PEVA skin. Such a device is easier to manufacture than those comprising multiple separated compartments but requires an excessive quantity of steroid.

Biodegradable microspheres have also been extensively used for local delivery of small molecules, drugs, peptides and proteins. In the so-called microspheres, the drug-containing core is surrounded by the polymeric matrix. In some systems, the drug is adsorbed or chemical conjugated on the surface of the polymer or entrapped into the core of the matrix. These morphological structures are sometimes mixed. For examples, in case of lipophilic drugs encapsulated into poly (lactic acid) (PLA) and poly(lactic/glycolic acid) (PLGA) microspheres, part of the drug is dissolved in the polymer but most is crystallized at the outer surface of the microspheres. In such circumstances diffusion of the drug is not possible.

Various active substance-release profiles can be achieved by adjusting the chemical composition and molecular weight (MW) of the active substance carrier, as well as the size and the porosity of the biodegradable microspheres and other factors (Li et al, Polymer for Advanced Technologies 2003, 14, 239-244 and references 7-10 within).

Mechanism by which suspended or dissolved drugs are released from biodegradable microspheres depends on different parameters including drug solubility, diffusion of drug from the microspheres, hydrolysis and weight loss of the polymer of the microsphere. The release profile is usually characterised by a an initial release phase (due to dissolution of drug particles present on the surface or drug particles having access to the surface via micropores of the microspheres). This release is affected by the drug solubility, drug loading as well as porosity and density of the microspheres. The subsequent release depends on the hydrolysis of the polymer and dissolution of the soluble oligomers to create pores/channels for drug diffusion. The polymer properties will influence the onset, duration and level of drug achieved during this phase.

Microspheres are usually administrated by subcutaneous or intra-muscular injection using a syringe with a fine needle. Duration of the drug release is mainly dependent of the physicochemical characteristics of the polymer used as drug carriers. Typical, depot of PLGA and PLA microspheres are used for the delivery of over 1-3 months. For longer time delivery or for delivery in a specific body part (with specific anatomical shape or mechanical stress) microspheres alone are not useful and an implant with specific geometry and mechanical properties is required.

To be implantable, the microspheres have to be structured in a specific 3D structure or matrix. For example, active drug delivery system releasing levonorgestrel have been prepared by compression molding of levonorgestrel-loaded polylactide and copolymers of lactic and glycolic acids microspheres prepared by solvent evaporation technique (Dinarvand R. et al, Drug Delivery Systems and Sciences 2001, 1, 113-116). However, the release profile of this matrix did not follow a Fickian model of kinetics and the use of hard conditions for compression molding (120 min at 90° C.) can induce either partial degradation of the polymer (processing temperature above Tg) or partial denaturation/inactivation of the encapsulated drug. Moreover, the mechanical properties of this kind of implant are expected to be insufficient as both polylactide and copolymers of lactic and glycolic acids have mechanical limitations. Another subdermal implant called Capronor uses poly(epsilon-caprolactone) and grain like pellets using fused cholesterol as matrix. Capronor II consists of 2 rods of poly(epsilon-caprolactone) each containing 18 mg of levonorgestrel. Capronor III is a single capsule of copolymer (caprolactone and trimethylenecarbonate) filled with 32 mg of levonorgestrel which has been developed to release the drug and biodegrades more rapidly than Capronor II. With both systems, the implant remains intact during the first year of use, thus could be removed if needed. Over the second year, it biodegrades to carbon dioxide and water, which are absorbed by the body. So, the controlled release is in that case only regulated by the chemical composition of the biodegradable polymeric microspheres without any regulation from the embedding matrix (fused cholesterol).

Hydrogels are one of the upcoming classes of polymer-based active substance delivery system due to biocompatibility and water permeation properties. By biocompatibility one means that the material does not induce any toxicity or immune reaction.

A wide range of hydrophilic polymers can be used to fabricate such hydrogels including natural or synthetic polymers and combination of both (see Hoffman et al. Adv Drug Del Rev 2002, 43, 3-12, J L Drury et al. Biomaterials 2003, 24, 43374351 for a review). Conventionally prepared by cross-linking hydrophilic polymers, hydrogels have the ability to absorb >20% of their weight of water while maintaining a distinct 3D structure. Swelling behavior of hydrogels is thus one of their important characteristics in relation with their use for pharmaceutical and biomedical applications since the equilibrium degree of swelling will influence (1) solute coefficient diffusion through the hydrogel, (2) surface and optical properties (especially in relation with their uses as contact lens), and (3) mechanical properties. Because of their high swelling capacity, release of low molecular weight (MW) water soluble drugs from hydrogels is relatively fast and difficult to regulate. In order to overcome the problem of rapid drug release, different following alternatives have been proposed.

Chemically immobilising the drug on the hydrogel matrix to form a polymer-drug conjugate has been discussed to prolong the drug action by the hydrolysis or biological scission of the covalent bonds (Sparer et al. in Controlled Release Delivery System, edited by T J Roseman and S Z Mansdorf, Marcel Dekker, New York, 1983, pp 107-119). However, covalent drug binding to macromolecular chains could inactivate the drug before its release.

Moreover, the amount of drug immobilization may be limited by the drug solubility.

Finally, an heterogeneous structure or composite hydrogel has been designed to retard drug release from hydrogels by encapsulation of the drug into hydrophobic domains. Yui et al described such devices based on lipidic microspheres (acting as drug microreservoirs) in degradable matrices of polyglycerol polyglycidylether crosslinked hyaluronic acid providing advantages such as regulating drug release from the biodegradable hydrogels, avoiding burst effect, and protecting drug from inactivation with the hydrophobic nature of the microreservoir. By the way, a zero-order release of lipidic microspheres was achieved in proportion to in vivo surface-controlled degradation of the crosslinked hyaluronic acid gels (Yui et al. J Control Rel 1993, 25, 133-143).

However, degradation is driven by an inflammation reaction due to hydroxyl radical by-products and the use of such a system for clinical application is questionable since the effect of this inflammation reaction on human health is not known. Moreover, this system may degrade too quickly.

An interpenetrating polymer network (IPN) of gelatin and dextran has been proposed as a dual-stimuli-responsive biodegradable hydrogel (Kurisawa M et al., J Control Rel 1998, 54, 191-200), wherein lipidic microspheres have been incorporated as drug-microreservoirs. The hydrogel, prepared below the sol-gel temperature, was found to release lipidic microspheres in the presence of both alpha-chymotrypsin and dextranase, whereas the release is hindered in the presence of either one enzyme only. However, this system is poorly-controlled since there is possible variation of enzyme content from patient to patient.

With the same prospect to retard leakage of entrapped agents from hydrogels, U.S. Pat. No. 6,632,457 describes a composite drug delivery system formed by dispersion of hydrophobic microdomains that can be made of oil, fat, fatty acid, wax, fluorocarbon, or other synthetic or natural water immiscible phase forming lipidic microspheres within an absorbable hydrogel. This system is suited for the controlled release of water soluble drugs having a relatively low MW, (having preferably a MW less than 2,000 daltons and a water solubility higher than 0.01 mg/ml) either alone or in combination. Suitable hydrogels described in this patent are absorbable hydrogels like those formed by addition polymerization of acrylic-terminated, water soluble chains of PLA-b-PEG-b-PLA triblock copolymers or cross-linking network comprising polypeptide or polyester components as the enzymatically or hydrolytically labile components.

Unfortunately, the mechanism by which the diffusion of the water soluble therapeutic compound is retarded is not understood, thereby making this system poorly predictable. This system is not suitable for the controlled release of drugs having a high MW and a poor water solubility and the residence time of the delivery system is limited (using bioresorbable/biodegradable polymers).

In addition, because of the biodegradable hydrogel, such a delivery system is also not appropriate for the controlled release of low MW drugs over a long period of time because the residence time of such a system is limited by the degradation rate of the polymer component. Finally, such a delivery device could also induce inflammatory reaction due to local acidification.

In view of the foregoing, there is a need for developing an implantable active substance delivery system that can be implanted in any part of the body and will allow the controlled and sustained release of active substances, whatever their physicochemical properties (water solubility, MW) and pharmacokinetics properties including active substance having limited diffusion capability (poorly water soluble and/or high MW).

It is an object of the invention to encounter the aforementioned drawbacks by providing a biocompatible delivery system, which is easily processable into a solid and specific 3D structure to fit the anatomy of the implantation site. The delivery system according to the invention is soft and elastic in the hydrated state for an easy insertion and an optimal comfort for the patient and is resistant to chemical or structural degradation (biostability) over the whole time of implantation to be finally removed at the end of use.

It has surprisingly been found that the active delivery system according to the invention exhibits a high swelling capacity and elastic properties in presence of water despite of the presence of microcarriers in the hydrogel matrix. The presence of microcarriers does not modify the swellability and elastic property of the hydrogel matrix.

By elastic property one means the tendency of a body to return to its original shape after it has been stretched or compressed. By swelling capacity one means the capacity of the hydrogel matrix to swell in presence of water. Both factors also contribute to the release rate of the active substances.

It is provided, according to the invention a solid active substance delivery system as indicated at first which is characterised in that the matrix consists in a biocompatible and biostable crosslinked covalent hydrogel, and the microcarriers are homogeneously embedded in the matrix and contain at least two active substances. The microcarriers are preferably microspheres in the size range of 1-1000 microns made of biodegradable and biocompatible (co)polymers.

Thus, said system according to the invention provides a biocompatible, biostable and easily processable active substance delivery system composed of a non-degradable hydrogel-type matrix (H) forming the core of said delivery system for a controlled and sustained release of any active substance or any combination of active substances whatever their water solubility and/or MW. It should be understood that composition and morphological characteristics of both biodegradable polymeric microcarriers and hydrogel-type matrix will be tuned to reach the desired release pattern of each individual active substance. Such biostable and biocompatible delivery system can be implemented in any part of the body.

The hydrogel matrix according to the invention is a cross-linked polymer network providing the delivery system with stability, elasticity, swelling and flexibility in the hydrated state.

Particularly the swelling capacity of the hydrogel matrix in the delivery system is comprised between 25 and 40% of its weight and its elastic modulus is comprised between 0.17 and 0.5 MTA in the hydrated state whereas its tensile strain at break is preferably between 1 to 7 MTA.

The hydrogel matrix is preferably made of polymers or copolymers allowing a regulation of the balance of hydrophilicity/hydrophobicity and being selected from the group consisting of (meth)acrylic polymers, poly(meth)acrylic acid, poly(hydroxy)alkyl(meth)acrylate, polyalkoxyalkyl methacrylate, poly(meth)acrylamide, polyvinylpyrrolidone, polyethyleneglycol and hydrophilic polyurethanes.

Indeed copolymerization of different hydrophilic/hydrophobic co-monomers is a way to tune the swelling behavior of synthetic hydrogels. For examples, small amounts of methacrylic acid (MAA) as a comonomer can dramatically increase the swelling of poly(hydroxyethylmethacrylate) (PHEMA). On the contrary, the hydrophobic nature of methyl methacrylate (MMA), allows copolymers of methyl methacrylate and hydroxyethylmethacrylate (HEMA) to exhibit a lower degree of swelling than pure PHEMA.

In a particularly preferred embodiment according to the invention, the hydrogel matrix is made of poly(hydroxy)methylacrylate or copolymer of (hydroxy)methylacrylate and methylmethacrylate and the polymerization reaction is carried under mild conditions using redox initiators to avoid any damage to microcarriers during the synthesis of the hydrogel-type matrix. Preferably, the hydrogel is synthesized at a temperature lower than the melting temperature (Tm) of the polymer microcarriers. Preferably, the synthesis temperature of the hydrogel-type matrix is lower than 59° C. when microcarriers are made of poly(epsilon-caprolactone).

In another particularly preferred embodiment according to the invention and when microcarriers are made of amorphous (co-)polymers, the hydrogel-type matrix is synthesized at a temperature lower than the glass temperature (Tg) of the microcarriers Preferably, the synthesis temperature of the hydrogel-type matrix is lower than 57° C. for poly(D,L-Lactide) microcarriers.

Amorphous (co)polymers are for example random copolymers of lactic and glycolic acids (PLGA).

The microcarriers according to the invention are biodegradable microspheres or microcapsules, homogeneously distributed into the hydrogel matrix. They contribute in combination with the hydrogel to the release rate regulation of the active substances contained therein.

Microcapsules are microparticles of any shape.

Microspheres (msp) are fine spherical particles with a diameter preferably in the range 1 to 1000 microns.

Microcarriers are made of biodegradable polymer or co-polymer. Such (co)polymers can be natural or synthetic polymers. By natural polymers one means (1) polypeptides and proteins like albumin, fibrinogen, gelatin, and collagen, (2) polysaccharides like hyaluronic acid, starch and chitosan. By synthetic polymers, one means for example aliphatic polyesters (homo- and copolymers), polyhydroxyalkanoates, polyanhydrides, poly(orthoesters), polyphosphazenes, poly(alkylcyanoacrylate), poly(amino acids) and the like

Aliphatic polyesters are for example poly(lactic acid) (PLA), poly(glycolic acid) (PGA), poly(lactic/glycolic acids) (PLGA), poly(hydroxybutyric acid) (PHB), poly(epsilon-caprolactone) (PCL) homopolymers and any copolymers of lactic acid, glycolic acid with epsilon-caprolactone; poly(orthoesters), poly(alkylcarbonates), poly(amino acids), polyanhydrides, polyacrylamides poly(alkylcyanoacrylates) and the like.

Microcarriers are preferably made of Aliphatic polyesters, such as poly (lactic acid) (PLA), poly(epsilon-caprolactone) (PCL) and copolymers of lactic and glycolic acids (PLGA). They are used as microencapsulating material for both lipophilic or hydrophilic drugs. These synthetic biodegradable polymers are highly hydrophobic and dissolve in organic solvents in which lipophilic drugs are soluble and hydrophilic drugs can be suspended or emulsified as an aqueous solution to prepare microspheres with the drug encapsulated.

Usual methods to prepare microspheres are (1) emulsion solvent evaporation (O/W, W/O, and W/O/W emulsion evaporation where O stands for oil and W for water phase), (2) phase separation (nonsolvent addition and solvent partitioning), (3) interfacial polymerization and (4) spray-drying.

The method to prepare drug encapsulation by microspheres is wellknown in the art. Various microencapsulation techniques incorporating active substances into a polymer are cited in U.S. Pat. No. 5,665,428.

In an embodiment according to the invention, the active substance delivery system comprises at least two populations of microcarriers. As already mentioned above, by microcarriers, one means microparticles, or microspheres made of biodegradable and biocompatible polymers. Different populations of microcarriers mean (1) microspheres made of different polymers, (2) microcarriers made of the same polymer but having different molecular weights, (3) microcarriers made of the same polymer but having different sizes.

In addition, the active substance delivery system could comprise at least two populations of microcarriers, each population containing an active substance different from the active substance contained in another population.

Moreover each population of the active substance delivery system according to the invention can be either made of a biodegradable (co)polymer which is different from or identical to the biodegradable (co)polymer forming the other population.

Thanks to these features, the active substance delivery system allows the delivery of various active substances, the delivery system according to the invention comprising one or more different populations of active substance-loaded biodegradable polymeric microcarriers (BPM). Multiple drug administration is thus possible using the same or different populations of biodegradable polymeric microcarriers (BPM) capable of releasing active substance at different rates by degradation and/or diffusion-based release mechanisms. Therefore, the release rate of each individual drug can be programmed by appropriate modification of both microcarriers and hydrogel matrix.

Mechanism by which the active substance is released from the biodegradable microcarriers further depends of drug solubility, diffusion of drug from the microspheres, hydrolysis and weight loss of the polymer of the microsphere.

The drug is released by diffusion through the pores or channels of the polymeric matrix, by diffusion across the polymer barrier or by erosion of the polymer barrier of the microcarrier. Usually diffusion and erosion can be concomitant and the relative contribution of these two phenomena depends on the polymeric composition of the microcarrier

Biodegradable aliphatic polyesters, like poly(lactic acid) (PLA), poly(glycolic acid) (PGA), poly(epsilon-caprolactone) (PCL) homopolymers and any copolymers of lactic acid, glycolic acid, epsilon-caprolactone will be used as microcarrier for the encapsulating materials thank to their biocompatibility, easily processability, and most interestingly, the possibility to tune their macromolecular characteristics and thus their degradation rate, permeability and release rate properties by appropriate synthetic routes. These macromolecular characteristics including polymer MW, crystallinity (from amorphous to semi-crystalline), and (in case of copolymers) ratio of the comonomer can be properly tuned as a result of their synthesis through a living ring opening polymerization mechanism Most of these (co)-polyester are commercially available and FDA approved for clinical use in Humans. Low-molecular weight polymers (<20,000) are prepared by direct condensation of the lactic and/or glycolic acids without catalyst. High-molecular weight polymers are produced by the ring opening polymerization with catalyst such as dialkyl zinc, trialkyl aluminum, and tetraalkyl tin in which lactide and/or glycolide cyclic dimers are (co)-polymerized.

Polymers synthesized using living ring opening polymerization mechanisms will be preferred because of the possibility to finely tune the chemical composition and macromolecular architecture of the polymer as well as the polymer molecular weight and polymolecularity as a result of the macromolecular engineering.

For fast release (1-3 months), PLGA copolymers with LA/GA ratio from 100/0 to 25/75) will be used. The lower the molecular weight of the polymer, the faster the degradation rate and release of the drug by erosion of the microparticles. For longer release period (up to 18 months), more hydrophobic polymer like PCL will be preferred.

Any homo- and co-polymers formed by any combinations of L-Lactide, D,L-lactide, glycolide, epsilon-caprolactone, trimethylene carbonate and dioxanone can also be used to fit the desired degradation rate. Interestingly, poly(lactic acid)-poly(epsilon-caprolactone) copolymers can be designed that shown a double release mechanism: diffusion-based release due to highly permeable but slowly degradable poly(epsilon-caprolactone) segment and erosion-based release due highly degradable poly(lactic acid) block.

Once synthesized, microspheres will be preferably embedded into the hydrogel by dispersion of the solid microspheres into the solution precursor of the hydrogel. The composition of the hydrogel further provides long-term stability, resistance and flexibility, allowing the system according to the invention for being comfortably implantable. The hydrolytic degradation of microspheres may be up-regulated by the equilibrium water content of the hydrogel-type matrix (depending on its swelling capacity) which can in turn be controlled by adjusting the hydrophilic/hydrophobic balance, crosslinking density (mesh size) of the hydrogel network, or the like.

According to the above characteristics, said system offers the possibility to tailor the release profile of active substances by combining multiple release mechanisms in the same device:

(1) diffusion through/erosion (degradation) of biodegradable polymeric microcarrier walls

(2) diffusion through the hydrogel-type matrix porous network, in relation with its swelling capacity.

Advantageously, the active substance delivery system according to the invention further comprises a release rate modifier in the hydrogel matrix and/or in the microcarriers.

Because drug release from biodegradable microcarrier associated-hydrogel matrix can be too fast, release rate modifier can be added both in the biodegradable polymeric microcarrier or in hydrogel matrix. Use of release rate modifier has been reported to act as encapsulating materials. Release rate modifier are for example nanoclays

According to the invention, the active substance is a substance having a pharmaceutical, therapeutic, physiological or biological effect. Said delivery system is a system for being applied locally in/on a human or animal body or to be used as substrate for cell or tissue culture or engineering.

As mentioned herein, the terms “a poorly water soluble substance” refer to a substance having a low saturation solubility. An example of a poorly water-soluble drug used in gynecology is the levonorgestrel which presents a saturation solubility of 5 μg/ml at 37° C. It should be mentioned that the levonorgestrel is one of the less water-soluble steroids. Other examples of drugs with moderate lipophilicity are dexamethazone and timolol maleate salt frequently used in opthamology.

In a particularly preferred embodiment one population of microcarrier contains a steroid hormone and another population of microcarriers contains an inhibitor of matrix metalloproteinase.

Such system is thus designed for a local (intravaginal or intrauterine) co-administration, of both a poorly water soluble steroid hormone like Levonorgestrel and an inhibitor of matrix metalloproteinase (MMPi), for suppression of uterine abnormal bleeding during contraceptive treatment.

It can also be used for the delivery of any steroids, hormones and hormones agonistic or anti-agonistic or a combination thereof.

The system can also deliver, individually or simultaneously to the steroid, any other biologically-active agents like antiviral, antibacterial, antiparasitical, antifungical, anti-inflammatory, antitumoral, or antineoplastic activity as well as analgesic agents, and agents protecting against HIV and others sexually transmitted diseases.

The invention also relates to the use of the active substance delivery system according to the invention as intra-articular, intra-muscular, intra-mammary, intraperitoneal, subcutaneous, epidural, intra-ocular, conjunctival, intrarectal, intravaginal, intracervical, intrauterin or any implantable delivery system.

Moreover, the invention relates to the use of the active substance delivery system according to the invention as cell culture, tissue engineering, in particular, cartilage, skin, bone, muscles or the like tissue engineering, and regenerative medicine support device.

Furthermore, the invention relates to the use of the active substance delivery system according to the invention as DNA or protein delivery system, in particular, in a gene therapy or in a therapy requiring the direct delivery of proteins.

Advantageously, this active substance delivery system can be used for the delivery of oligopeptide active substances, cytokines, tissue-specific growth factors, protein-based growth factors or any molecules that can induce differentiation of endogenous or transplanted progenitor cells into the appropriate cell types, and can be used in the healing, reparation or regeneration of diseased or failed tissues and/or organs. The release of plasmid or non-viral DNA encoding for therapeutic or tissue inductive protein represents a promising alternative to the direct delivery of proteins.

Other embodiments of the device according to the invention are mentioned in the annexed claims.

As above indicated, the present invention relates to an implantable active substance delivery system composed of biodegradable and biocompatible polymeric microcarriers (BPM) dispersed into a soft and elastic hydrogel matrix (H) for the controlled release of preferably two or more active ingredients, at different rates over a long period of time. Multiple active substance administration is possible using different populations of biodegradable polymeric microcarriers capable of releasing active substances at different rates by degradation and/or diffusion-based release mechanisms. Therefore, the release rate of each individual active substance can be programmed by appropriate modification of both microcarriers and hydrogel matrix. A preferred application of such a device is the local co-administration (intrauterine or intravaginal) of both a poorly soluble steroid hormone like levonorgestrel (LNG) and a inhibitor of matrix metalloproteinase (MMPi). So it is possible to obtain a specific therapeutic dose and release profile for the suppression of abnormal uterine bleeding during contraceptive treatment.

The system according to the present invention is a long-term active substance delivery system comprising biodegradable polymeric microcarriers for the independent and controlled release of one, two or more therapeutic molecules embedded into a biostable hydrogel matrix providing long-term stability, resistance and flexibility.

The delivery system according to the invention offers the possibility to tailor the release profile of active substances by combining multiple release mechanisms in the same device:

(1) diffusion through/erosion (degradation) of biodegradable polymeric microcarrier walls

(2) diffusion through the hydrogel-type matrix porous network, in relation with its swelling behaviour.

Thus the system is not restricted to relatively low molecular weight neither to relatively water soluble active substances. As the system can be composed of different populations of biodegradable polymeric microcarriers, it can be used for the controlled release of two or more active substances whatever their physico-chemical properties. The polymers used as active substance carriers (biodegradable polymeric microcarrier) are biodegradable synthetic polymers but preferentially aliphatic polyesters whose main degradation mechanism is driven by hydrolytic scission of the esters covalent bonds. The system is thus highly versatile since numerous parameters can be modified in order to adjust the release rate of any active substance, independently, by playing on:
biodegradable polymeric microcarriers chemical composition: e.g. molecular weight (MW), MW distribution, crystallinity and end-group chemistry of the polymer, and (in case of copolymers) structure and co-monomers ratio,
biodegradable polymeric microcarriers properties: surface porosity, average size and size distribution
active substance loading in the biodegradable polymeric microcarriers

Moreover, the hydrolytic degradation of such polymers may be up-regulated by the equilibrium water content of the hydrogel matrix (depending on its swelling capacity) which can, on its turn, be controlled by adjusting the hydrophilic/hydrophobic balance, the crosslinking density (mesh size) of the hydrogel network, or the like.

Additionally, the release rate of the active substances can be tuned by dispersion of release rate modifier (RRM) used as fillers either in one or in both biodegradable microcarriers and hydrogel-type matrix phases as described below.

Because of the possibility to tune the hydrophilicity/hydrophobicity balance by copolymerisation, acrylic polymers, selected in the group comprising methylmethacrylate (MMA), hydroxyethylmethacrylate (HEMA), ethylmethacrylate (EMA), phenylethyl(meth)acrylate (PE(M)A), can be used for the preparation of the implant. These polymers are known for being biocompatible and for having a long term use as contact lenses and intraocular implants. Other polymers selected in the group comprising poly(meth)acrylic acid, polyacrylamide and poly(1-vinyl 2-pyrolidone), polyethyleneglycol, hydrophilic polyurethane, can also be used.

Most preferentially, implants will be composed of PHEMA (poly(hydroxyethylmethacrylate)) combining biostability over the whole implantation time and a relatively low modulus (stiffness) for greater comfort. Suitable hydrogels should exhibit mechanical properties in the range of 0.17-0.5 MPA for the elastic modulus in the hydrated state and 1-7 for the tensile strain at break.

Many different routes have been used to synthetise both physical and chemical hydrogels as described in paper reviews by Hoffman et al (Hoffman et al. Adv Drug Del Rev 2002, 43, 3-12). A general review over preparation and properties of PHEMA (poly(hydroxyethylmethacrylate)) hydrogels is given by Horak D et al. (Horak et al. PBM Series 2003, 1, 65-107). Chemical crosslinking will be preferred over physical crosslinking to create hydrogels with good mechanical stability. Chemical gels will be preferably formed by copolymerisation of a monomer and a crosslinker in bulk or in aqueous solution for example HEMA+EGDMA in water (hydroxyethylmethacrylate+ethyleneglycoldimethacrylate, in water). As an alternative to EGDMA, 4-{(E)-[(3Z)-3-(4-(acryloyloxy)benzylidene)-2-hexylidene]methyl}phenyl acrylate may be used. As another alternative, monomers can be copolymerized with macromers (e.g. HEMA+PEGDMA (hydroxyethylmethacrylate+poly(ethyleneglycoldimethacrylate)) or with a water soluble polymer or in the presence or not of a crosslinker. Polymers can be directly crosslinked in bulk or in solution using radiation, chemical crosslinker or multifunctional reactive compounds. Finally monomers can be polymerized within a different solid polymer to form an interpenetrating polymer network (IPN) gel. The conditions for the hydrogel synthesis may be chosen to avoid any damage or degradation of the pre-formed polymeric microcarriers. For examples, the hydrogel synthesis using redox initiator system is exothermic. So there is a risk of melting of the polymeric microcarriers especially those made of polymer with relatively low melting temperature (Tm) (poly(epsilon-caprolactone, Tm=59° C.). To avoid any damage to microcarriers during hydrogel synthesis the temperature of the reaction has to be maintained below Tm of the polymeric microcarriers and preferentially below 59° C. in the case of poly(epsilon-caprolactone) microcarriers. Biodegradable polymeric microcarrier solubilization can also be avoided by using crosslinked polymer for the preparation of the microcarriers like for examples poly(epsilon-caprolactone)-diacrylate polymer.

Photopolymerization using UV initiator with very short polymerization time (1-3 min) can be also used as described in the international application WO 9603666.

Other radiation techniques can also be used for preparation of hydrogel by copolymerisation of HEMA (hydroxyethylmethacrylate) with PEG-MA (polyethyleneglycolmethacrylate) at very low temperature (Kwon O H et al., J of Industrial and Engineering Chemistry 2003, 9(2), 138-145 Bhattacharya A et al, Prog. Polym. Sci. 2000, 25, 371-401, pp. 375-383).

Various microencapsulation techniques incorporating active substances into a polymer are cited in U.S. Pat. No. 5,665,428. The choice of the microencapsulating method mostly depends on the active substance solubility. Immobilization of lipophilic active substance within a hydrophobic polymer like poly(lactic acid), poly(lactic-glycolic acid), or the like, is easily carried out by the conventional oil/water emulsion-evaporation.

For encapsulation of protein or peptides, which are hydrophilic active substances, different methods have been described such a non-aqueous phase separation technique, i.e oil/oil emulsion followed by solidification of the internal phase. Peptides and proteins are also efficiently encapsulated by a modified solvent-evaporation method based on double water/oil/water emulsion (U.S. Pat. No. 4,652,441) and by a phase separation or coacervation process.

For long-acting controlled release of contraceptives, microspheres were prepared from block copolymers of epsilon-caprolactone and D,L-Lactide using solvent-evaporation process. The same kind of copolymers has been used for the controlled release of progesterone and estradiol over 40 days.

Alternative routes for microencapsulation can be:

a) polymer melt process as described in U.S. Pat. No. 5,665,428. However, for being applicable to heat sensitive peptide and protein active substances, this system is limited to copolymers which can be processed into microcarriers at temperature below 100° C.

b) supercritical CO₂ technology by both SAS (Supercritical Anti-Solvent) (Bertucco A. et al., Process Technology Proceedings (1996), 12 (High Pressure Chemical Engineering), 217-222) or RESS (Rapid Expansion of Supercritical Solutions) methods (Tom, Jean W et al. ACS Symposium Series (1993), 514 (Supercritical Fluid Engineering Science), 238-57). The main advantage of this solvent-free process is the absence of toxic and leachable residues that could be difficult to remove, or could induce active substance denaturation/degradation.

Microspheres and hydrogel will be combined as follows: Once formed, washed and dried, active substance-loaded biodegradable polymeric microcarriers will be dispersed into the gel-precursor solution following by gelation of the dispersion by using any of the methods previously described and preferentially by chemical crosslinking of acrylic monomers.

Because active substance released from BM associated-hydrogel matrix can be too fast, release rate modifier can be added both in the biodegradable polymeric microcarrier or in hydrogel matrix. The use of releaser rate modifying agents has been reported in the literature (see U.S. Pat. No. 6,632,457) as encapsulating material but the mechanism by which they retard the active substance release is still unclear. Preferentially, nanofillers could be used that can be easily dispersed into many different polymer matrix and in order to modify their transport properties, even at very low loading (<1 wt %). Because of their high shape ratio and submicrometric size, they can considerably increase the tortuosity factor resulting in an increase of the active substance diffusion pathway. Such nanofillers, including for example the nano-clay cloisite 30B, or the like, can be used either as a modifier in the biodegradable polymer microcarriers to control the degradation behavior and hydrophilicity and/or as a modifier of the hydrogel matrix to change the active substance diffusion pathway by increasing the tortuosity factor of the active substance carrier.

The system described in this patent can be used to deliver any therapeutic molecules whatever its physico-chemical and pharmacokinetics properties (i.e. active substance MW and water solubility). It is not restricted to relatively low MW, water soluble active substances like in the system described in U.S. Pat. No. 6,632,457. Because different populations of microcarriers (biodegradable polymeric microcarrier) can be incorporated into the hydrogel-core, this system can be used or deliver any combination of one or more biologically-, physiologically-, or pharmaceutically-active ingredients that have to be delivered at different rates over a relatively long period of time.

These release systems are preferably designed for a local (intravaginal or intrauterine) co-administration, of both a poorly water soluble steroid hormone like Levonorgestrel and an inhibitor of matrix metalloproteinase (MMPi), for suppression of uterine abnormal bleeding during contraceptive treatment. It can also be used for the delivery of any steroids, hormones, hormones agonist or anti-agonist, or a combination thereof. The system can also deliver, individually or simultaneously to the steroid, any other biologically-active agents like antiviral, antibacterial, antiparasitical, antifungical, anti-inflammatory, antitumoral, or antineoplastic activity as well as analgesic agents, spermicidal agents and agents protecting against HIV and others sexually transmitted diseases.

Other molecules of interests for different applications include agents affecting the central nervous system, metabolism, respiratory or digestive organs, antiallergenic agents, cardiovascular agents, hormone preparations, antitumoral agents, antibiotics, chemotherapeutics, antimicrobials, local anaesthetics, antihistaminics, vitamins, antifungal agents, vasodilatators, hypotensive agents, immunosuppressants.

Alternatively, this active substance delivery system can be used for the delivery of oligopeptide active substances, cytokines, tissue-specific growth factors, protein-based growth factors or any molecules that can induce differentiation of endogenous or transplanted progenitor cells into the appropriate cell types, and can be used in the healing, reparation or regeneration of diseased or failed tissues and/or organs. The release of plasmid or non-viral DNA encoding for therapeutic or tissue inductive protein represents a promising alternative to the direct delivery of proteins.

These hydrogel-based active substance delivery systems are particularly suitable for applications in gynecology and opthamology.

For ophthalmic applications, a major concern is the high sensitivity of the ocular tissues (e.g. the retina) to drugs and especially to newer therapeutics agents such as those developed from proteomics and gene therapy. They are expected to heighten the need for optimal drug delivery both in time and to specific sites. The invention allows to achieve site specific delivery and more favorable retention time in the eye together with reducing incidence of toxicity or side effects (no burst effect). This burst release could indeed endanger intraocular tissues in the immediate postoperative period.

In opthamology, each specific medical application imparts restrictions on the size and shape of the implant which must be miniaturized and “gel” soft for easy insertion and minimal trauma to adjacent tissues. The hydrogel-matrix based delivery system can be easy micro-machined into micro-devices, while displaying soft aspect preventing tissues damages.

In case of intraocular controlled drug release inserts, envisioned locations are subconjunctival, intravitreal, endocapsular, suprascleral, in a buckle groove, and over a melanoma.

Different pathologies can be concerned, such as glaucoma, uveitis, wound healing, herpes simplex, . . . and even immune response modulation.

Because they can be processed to have many different physical forms including (a) solid moulded or post-machined (lathe cutting) forms, (b) membranes, sheets, or the like, they can be used for local delivery using different possible routes of administration including intra-articular, intramuscular, intra-mammary, intraperitoneal, subcutaneous, epidural, intra-ocular, intrarectal, intravaginal, intrauterin, etc.

Active substance delivery system according to the present invention can therefore be used as a sub-cutaneous, intramuscular or intra-peritoneal implant or in different organs or tissues such as join, muscle, breast, eye, vagina, uterus, and the like. Besides applications in active substance delivery, these systems can also be useful for cell culture, tissue engineering (eg. cartilage, skin, bone, muscles, or the like), regenerative medicine, as well as gene therapy.

### EXAMPLES

### Example 1

### Manufacturing of Hydrogel Containing Blank poly(L-lactide) (PLLA) Microspheres

In this example, biodegradable polymeric microspheres have been prepared using a W/O/W (water/oil/water) emulsion-evaporation technique as follows: 1 g of PLLA (Boerhinger-Ingelheim) was dissolved in 10 ml of dichloromethane under magnetic stirring. An aqueous gelatine solution was prepared by dissolving 1 g of gelatine in 5 ml of deionized water at 40° C. The gelatine solution was added to the polymer solution and the mixture was emulsified by sonication using an Ultraturax at 13500 rpm for 2 min in a Falcon tube. The resulting primary w/o emulsion was then injected drop-by-drop with a micropipette to 100 ml of a 2 wt % aqueous solution of polyvinylalcohol (PVA) contained in a 250 ml cylindrical glass flask at 10° C. The resulting w/o/w emulsion underwent mechanical stirring and the solvent was allowed to evaporate first at 10° C. for 30 min then at 30° C. for 90 min. The resulting solid microspheres were collected after filtration and washed three times with deionized water before being freeze-dried. The surface morphology of the microspheres was examined by SEM (Jeol JSM-840A) after platinum coating. Microspheres, having a size ranging from 100 to 300 μm and a porous structure, were collected.

A monomer solution was prepared by mixing 50 ml of purified hydroxyethylmethacrylate (HEMA), 3 g of dimethylaminoethyl methacrylate (MADAM) and 0.05 g of ethyleneglycoldimethacrylate (EGDMA). 0.25 g of ammonium persulfate ((NH₄)₂S₂O₈) and 0.1 g of sodium metabisulfite (Na₂S₂O₅) were dissolved in 30 ml of water to form a solution of redox initiator agent. This solution was added to the monomer solution with a 1/3 volume ratio in a cylindrical plastic mould. The mixture was homogenised under magnetic stirring and refrigerated at 10° C. prior to the addition of microspheres. A given amount of the pre-formed biodegradable microspheres was added the solution and the hydrogel was synthesised at 10° C. to avoid any damage to the microspheres. Polymerization of the hydrogel was observed after about 30 min. Gentle agitation of the dispersion allowed the microspheres to be homogeneously distributed into the hydrogel matrix. The relatively optical transparency of the hydrogel matrix allowed the biodegradable polymeric microspheres to be easily visualised. The biodegradable polymeric microspheres may contain any of the therapeutic ingredients as described here above.

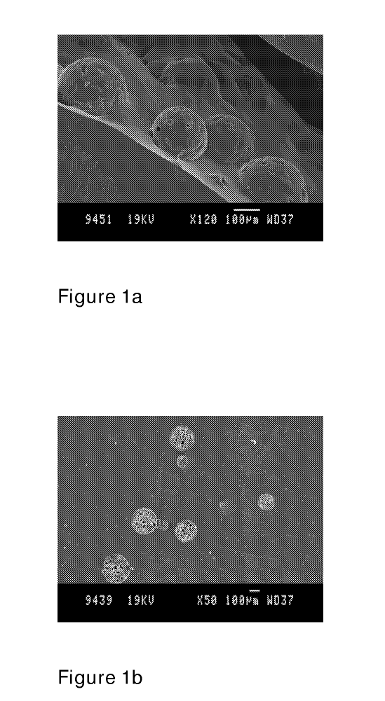

Referring to FIG. 1, biodegradable polymeric microspheres are properly dispersed into the hydrogel matrix, with preservation of their structure.

The FIG. 1 represents a SEM micrograph of a biodegradable microsphere-loaded poly(hydroxyethylmethacrylate) hydrogel matrix, showing (a) the surface of the hydrogel and the morphology of the biodegradable microspheres and (b) the cross-section of the hydrogel showing the dispersion of the biodegradable microspheres and their internal morphology.

The W/O/W (water/oil/water) process can be used for encapsulation of water-soluble active substances including peptides or proteins. In the case of hydrophobic active substances (i.e. steroids and the like), simple O/W (oil/water) emulsion-based process are preferred. PLLA (poly(L-lactide) microspheres prepared using this simple method have a size within the same range (100-300 μm) but with lower size polydispersity.

Alternatively, hydrogels containing blank poly(D,L-lactide) microspheres can be synthesised using the same process as described in example 1. Amorphous PDLLA (poly(D,L-lactide) are known to degrade faster than semi-crystalline PLLA (poly(L-lactide) microspheres.

### Example 2

### Formation of Hydrogel Containing Blank poly(PCL) (poly(poly-(ε-caprolactone) Microspheres

The procedure given in the previous example is repeated, but by substituting poly(L-lactide) microspheres with poly(PCL) ones. (Poly-(ε-caprolactone) microspheres are prepared according to the same recipe as for poly(L-lactide) carriers. As an alternative to emulsion-based process, polymeric microspheres can also be prepared by other routes as reported previously; the choice of the microencapsulation technique being mainly dictated by the properties of the active substance.

### Example 3

### Release of the Levonorgestrel from the PHEMA Hydrogel Matrix

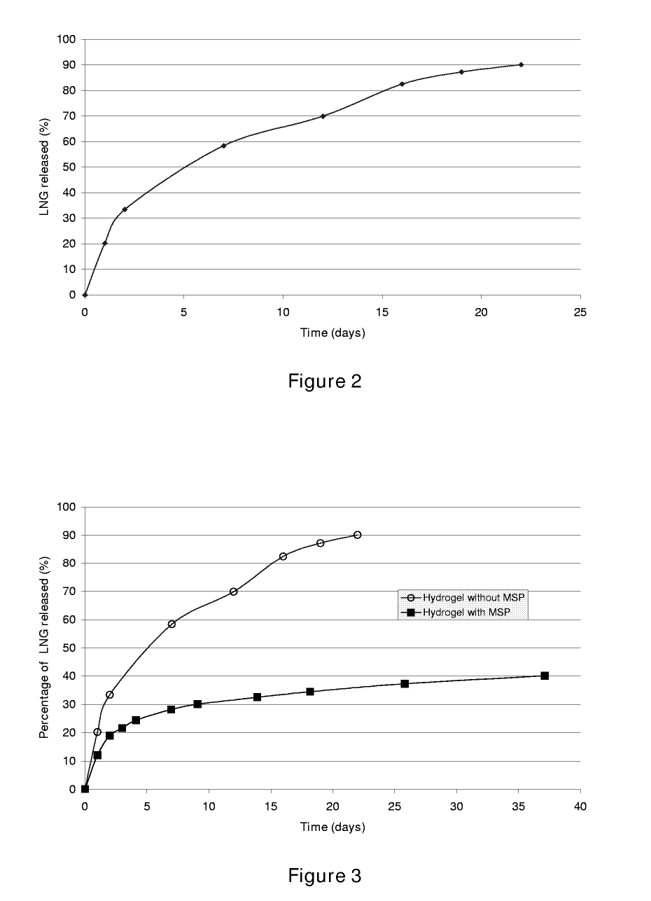

A stock monomer solution was prepared by mixing 10 ml of hydroxyethylmethacrylate (HEMA) and 11 μl of ethyleneglycoldimethacrylate (EGDMA) (0.1 wt % of crosslinker). 1.5 ml of this stock monomer solution was collected and 2.5 mg of LNG was dissolved in it. N2 was bubbling in this solution for 5 min. before addition of 0.5 ml of initiator solution. The initiator solution was freshly prepared by mixing solutions of 6.5 mg/ml of potassium persulfate ((NH₄)₂S₂O₈) and of 3.2 mg/ml of sodium metabisulfite (Na₂S₂O₅) in water. After bubbling for 5 min into nitrogen, this initiator solution was added to the monomer solution and very well mixed in a reaction tube at room temperature. 15 min N2 bubbling. The reaction tube is closed and let at room temperature. After reaching an appropriate viscosity, the mixture was transferred into the final mold for complete polymerization. A piece of 0.21 g of the crosslinked hydrogel matrix was immersed into the dissolution medium (purified water) at 37° C. under stirring in an oscillatory bath at 1400 rpm. At different time intervals, 2 ml of the dissolution medium was collected for determination of the LNG content by high performance liquid chromatography equipped with UV detector. The release profile expressed as the percentage of results of LNG released is shown in FIG. 2. After an initial burst of 20 wt % of the total LNG content during the first 24 hrs, the release rate tends to stabilize after 10 days to reach a value of around 10 μg/day. After 20 days of incubation, 90 wt % of the total LNG content is released from the hydrogel. The results show that the release of LNG from the PHEMA-hydrogel matrix is very fast.

### Example 4

### Comparison of LNG Released from a PHEMA Hydrogel and from PCL Microparticles Embedded in a PHEMA Hydrogel

FIG. 3 shows the comparison of release rate of LNG from the hydrogel matrix (see example 3) and from PCL microspheres embedded a PHEMA hydrogel matrix of the same composition (see example 5 for preparation of this microparticles-hydrogel matrix). This figure clearly shows that the encapsulation of LNG inside polymer microspheres allows for the retardation of the drug release. After 20 days of incubation, 35 wt % of the LNG content is released from the microspheres-containing hydrogel matrix, which is much lower than the LNG released from the pure hydrogel after the same incubation time. This confirms that encapsulation of LNG into polymeric microspheres allow to retard its release from the hydrogel matrix as compared to the direct release from the pure hydrogel, the confirming that the microspheres are creating a barrier against the drug diffusion.

### Example 5

### Release of Levonorgestrel (LNG) and Estradiol (EST) from a PHEMA Hydrogel Matrix after Encapsulation into PCL Microspheres

In this example, two kinds of active molecules, i.e. levonorgestrel and estradiol have been encapsulated into biodegradable polymeric microspheres made of PCL. Two different populations of microspheres have been prepared by using PCL with different molecular weight, i.e. PCL of MW: 50,000 and 10,000 for encapsulation of LNG and EST, respectively). The same O/W emulsion-evaporation technique was used for encapsulations of both drugs. For the encapsulation of the LNG, 1 g of PCL (Solvay Interox, Mw: 50000) was dissolved in 20 ml of dichloromethane under magnetic stirring. 200 mg of LNG was dissolved in this solution leading to a theoretical LNG content of 20 wt %. A 0.27 wt % aqueous solution of polyvinylalcohol was prepared. The organic polymer solution was added into the PVA aqueous solution drop-by-drop with a micropipette at room temperature under agitation (IKA-WERK RW:20) at 300 rpm to form the O/W emulsion. The solvent was allowed to evaporate at room temperature for 24 hrs. The resulting solid microspheres were collected after filtration and washed three times with deionized water before being freeze-dried. For encapsulation of estradiol, PCL with MW of 10000 was synthesized by ring opening polymerization using a tin catalyst (dibutylstanadioxepane) as a catalyst ( ). Microspheres of estradiol with a theoretical loading of 5 wt % were prepared using the same method as for encapsulation of LNG.

Microspheres were embedded into a PHEMA hydrogel matrix as follows: 1.5 ml of the stock monomer solution was collected and 2.5 mg of both kinds of msp was dispersed in it. After bubbling for 5 min., 0.5 ml of the initiator solution was added. The initiator solution was freshly prepared by mixing solutions of 6.5 mg/ml of potassium persulfate ((NH₄)₂S₂O₈) and of 3.2 mg/ml of sodium metabisulfite (Na₂S₂O₅) in water. After bubbling for 5 min into nitrogen, this initiator solution was added to the monomer solution and very well mixed in a reaction tube at room temperature. 15 min N2 bubbling. The reaction tube is closed and let at room temperature. After reaching an appropriate viscosity, the mixture was transferred into the final mold for complete polymerization.

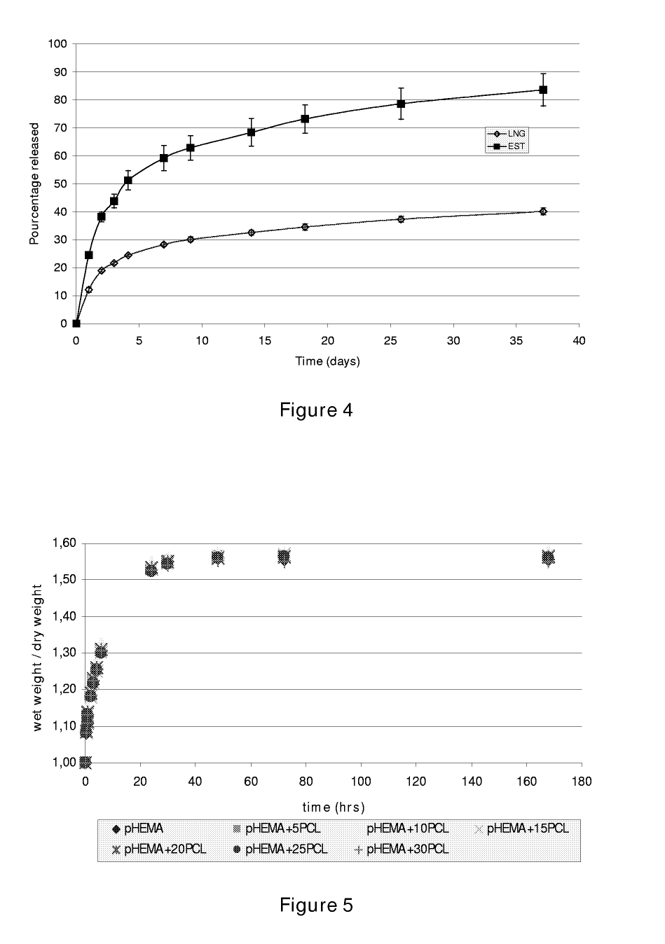

For the release experience, 0.33 g of the hydrogel was weighted and immersed into the dissolution medium (purified water) at 37° C. under stirring in an oscillatory bath at 1400 rpm. At different time intervals, 2 ml of the dissolution medium were collected for determination of both LNG and estradiol content by high performance liquid chromatography equipped with UV detector. The release profiles of LNG and EST from the hydrogel matrix are shown in FIG. 4. The diffusion rate of LNG is higher than that one of EST because of the higher loading of LNG as compared to estradiol (20% wt versus 5 wt %). After a burst release (10 wt % of the LNG content is released after 24 hrs), the release rate tends to stabilize after 7 days to reach a daily dose around 0.30-40 μg per day. The diffusion of EST is faster as compared to that one of LNG, most probably due to the lower initial content. Also the higher water solubility of the estradiol as compared to LNG may be responsible for a faster release and a higher initial burst. This example shows that release profile depends on different parameters including the drug solubility, the drug content and the microspheres properties.

### Example 6

### The Presence of the Microspheres does not Disturbed the Swelling Behaviour of the Hydrogel

The swelling behaviour was studied by immersion of the dry hydrogel samples in deionised water at room temperature. At certain time intervals, the hydrogel pieces were extracted from the water, blotted dry with a paper towel and weighed. The results obtained are depicted in the FIG. 5.

In FIG. 5 is illustrated the Dynamic swelling behaviour of pHEMA hydrogels incorporating different amounts of blank PCL microspheres at 5, 10, 15, 20, 25 and 30 mg microspheres/ml hydrogel.

From FIG. 5 it is visible that the loading of the hydrogel with microspheres—in the loading range investigated—does not have any major influence on the swelling behaviour of the pHEMA hydrogels containing PCL microspheres.

### Example 7

### Elastic Modulus Comparison for Hydrogel Matrix with Different Amount of Microspheres Embedded into the Hydrogel Matrix

In the present example a co-polymeric hydrogel matrix poly(hydroxy)ethylmethylacrylate (pHEMA) with 1% ethyleneglycoldimethacrylate (EGDMA) was prepared according to example 1.

PHEMA-based hydrogels exhibit a series of properties which make them preferential candidate building material for the core of the device:
ability to be synthesised at low temperature, using an initiator system with reduced toxicity
ability to be moulded in various shapes
good mechanical and water permeation properties, and good biocompatibility
monomer mixture able to act as a dispersing medium for the microspheres encapsulating the active principle, resulting intact microspheres uniform distributed in the hydrogel bulk
good mechanical properties such as elasticity and toughness, not affected by the presence of msp (up to 50 mg/ml of hydrogel)
capability to act as the second diffusion barrier of the active principle
   by copolymerization with hydrophobic monomer (MMA). Preliminary diffusion results suggest that the including of MMA in the hydrogel composition not only diminishes the water uptake (and consequently the hydration degree of the material, which in turn influences the transport of different molecules through hydrogel membranes), but also contributes to the retardation of LNG diffusion, probably due to the inclusion of glassy-like, low permeable regions into the hydrogel bulk.

The active principle, LNG, has a markedly hydrophobic character and a much higher solubility in the HEMA monomer than in water. Its solubility in the polymer is probably high enough to determine a high permeability of pHEMA membranes to LNG and consequently a fast diffusion of the active principle. It is also possible that, for high encapsulation percents of LNG in PCL microspheres, crystals of LNG lying on the microspheres surface become dissolved in the monomer mixture during hydrogel synthesis, leading to a molecular imprinting of the hydrogel matrix and improving the diffusion characteristics of LNG through the hydrogel matrix. Therefore, the including of the hydrophobic comonomer MMA in the hydrogel composition can help to modulate the diffusion of active principle, making the hydrogel the expected second diffusion barrier of the encapsulated LNG.

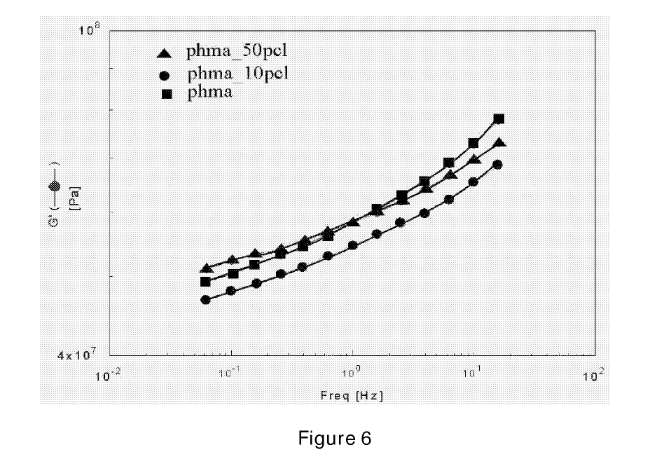

Elastic modulus for pHEMA hydrogel samples (1.0% EGDMA) without and with different microspheres amounts: 10 mg microspheres/ml hydrogel and 50 mg microspheres/ml hydrogel has been measured and is illustrated in FIG. 6.

It is observed that the presence of the microspheres did not modify the elastic properties of the hydrogel to a large extent.

Indeed modifications of the elastic modulus G′ appeared for hydrogels samples loaded with PCL microspheres as small and not-systematic, suggesting no contribution of the microspheres to the mechanical characteristics of the hydrogel bulk, as presented in FIG. 6.

The main physico-chemically properties of the hydrogels including swellability and elasticity are not affect by the presence of the microspheres at least in the loading range of 5-50 mg of msp/ml of hydrogel mixture. Even higher msp loading (up to 100 mg of msp/ml of hydrogel mixture) have shown to give similar observations.

Although the preferred embodiments of the invention have been disclosed for illustrative purpose, those skilled in the art will appreciate that various modifications, additions or substitutions are possible, without departing from the scope and spirit of the invention as disclosed in the accompanying claims.

1. A solid Active substance delivery system, comprising
a cross-linked hydrogel matrix, and
microcarriers which are embedded within the hydrogel matrix,
characterized in that the microcarriers are made of biocompatible and biodegradable (co)polymers, are homogeneously embedded into a biocompatible cross-linked hydrogel matrix and contain at least two active substances.

2. The active substance delivery system according to claim 1, wherein the hydrogel matrix has a swelling capacity in presence of water in the range of 25 to 40% of its weight.

3. The active substance delivery system according to claim 1, wherein the hydrogel matrix has a viscous modulus in the range of 0.17 to 0.5 MPA in the hydrated state and has a tensile strain at break between 1 and 7 MPA.

4. The active substance delivery system according to claim 1 wherein the hydrogel matrix is made of a polymer or copolymer selected from the group consisting of (meth)acrylic polymers, poly(meth)acrylic acid, poly(meth)acrylamide, polyvinylpyrrolidone, polyethyleneglycol and hydrophilic polyurethanes.

5. The active substance delivery system according to claim 4 wherein the hydrogel matrix is made of poly(hydroxy)methylacrylate.

6. The active substance delivery system according to claim 4 wherein the hydrogel matrix is made of poly(hydroxy)methylacrylate with ethyleneglycoldimethacrylate.

7. The active substance delivery system according to claim 1 wherein the hydrogel matrix is synthetised at a temperature lower than the melting temperature of the microcarriers.

8. The active substance delivery system according to claim 1 wherein the hydrogel matrix is synthetised at a temperature lower than the glass temperature of the microcarriers.

9. The active substance delivery system according to claim 1, wherein the microcarriers are microspheres in the size range of 1 to 1000 microns wherein the active substances are encapsulated.

10. The active substance delivery system according to claim 1 wherein the microcarriers are made of polymer or copolymer selected from the group consisting of collagen, glycosamyniglycans, chitosan, polyhydroxyalkanoates, aliphatic polyesters (homo- and copolymers), poly(anhydrides), polyphosphazenes, poly(alkylcyanoacrylate) and poly(amino acids).

11. The active substance delivery system according to claim 10 wherein the microcarriers are Aliphatic polyesters, selected from the group consisting of poly (lactic acid) (PLA), poly(epsilon-caprolactone) (PCL) and copolymers of lactic and glycolic acids (PLGA).

12. The active substance delivery system according to claim 1 wherein the different active substances are contained in different populations of microcarriers, each population containing an active substance different from the active substance contained in another population.

13. The active substance delivery system according to claim 1, further comprising a release rate modifier in the hydrogel matrix and/or in the microcarriers.

14. The active substance delivery system according to claim 1, wherein the active substance is a substance having a pharmaceutical, a therapeutical, a physiological or a biological effect.

15. The active substance delivery system according to claim 1 for use as drug delivery system locally on a human or animal body.

16. The active substance delivery system according to claim 15, for use as a sub-cutaneous, intra-muscular or intra-peritoneal implant or in an organ or tissue in human or animal.

17. The active substance delivery system according to claim 12, comprising one population of microcarrier containing an active substance x and another population of microcarriers containing another active substance y wherein x and y are selected from the group consisting of (a steroid hormone, an inhibitor of matrix metalloproteinase, an anti-angiogenic, an anti-inflammatory substance).

18. The process for making a solid active substance delivery system according to claim 1 comprising in step 1: dispersing a biodegradable (co)polymeric microcarrier containing at least two active substances into an hydrogel-forming matrix and in step 2: thereafter crosslinking said hydrogel-forming matrix by addition of an initiator characterized in that the microcarriers are homogeneously embedded into the biocompatible crosslinked hydrogel matrix.
